(19) United States
(12) Patent Application Publication
(10) Pub. No.: US 2008/0243309 A2
(43) Pub. Date: Oct. 2, 2008

(54) SYSTEMS FOR AND METHODS OF CHARACTERIZING REACTIONS

(75) Inventors: Jong Hong, Auburn, AL (US); Douglas Goodwin, Auburn, AL (US); Eduardus Duin, Auburn, AL (US); Sachin Jambovane, Auburn, AL (US); Robert Moore, Auburn, AL (US); Taek-Jeong Nam, Busan 608-041, KR (KR); Se-Kown Kim, Pusan 614-070 (KR)

Correspondence Address: HAVERSTOCK & OWENS LLP, 162 N WOLFE ROAD, SUNNYVALE, CA, 94086, UNITED STATES, 408-530-9700, JOWENS@HOLLP.COM

(73) Assignees: Auburn University, Auburn, AL (US); Pukyong National University, Pusan, 608-737 (KR)

(21) Appl. No.: 11/801,217

(22) Filed: May 8, 2007

(51) Int. Cl. G01N 33/53; G01N 33/50; G05B 21/00

(52) U.S. Cl. 700/266; 702/19

(57) ABSTRACT

An automated and computerized system for characterizing kinetic activities is disclosed. The system includes an optical unit with a controller chip. The controller chip has multiple reaction cells for simultaneously reacting samples of the catalyst under a range of reaction conditions and for optically monitoring the kinetic activity within each of the reaction cells. The system also preferably includes a temperature controller in thermal contact with the controller chip and an actuation device coupled to the controller chip for injecting and mixing samples of the catalyst with reagents into each of the reaction cells to form a product.

### RELATED APPLICATIONS

This Application claims priority under 35 U.S.C. § 119(e) from the Co-pending U.S. Provisional Patent Application Ser. No. 60/798,604, filed on May 8, 2006, and titled “MICROFLUIDIC CHIP FOR PROTEIN KINETICS,” and the Co-pending U.S. Provisional Patent Application Ser. No. 60/843,385, filed on Sep. 9, 2006, and titled “REACTION KINETIC LANDSCAPER,” the contents of which are both hereby incorporated by reference.

### FIELD OF THE INVENTION

This invention relates generally to systems for and methods of characterizing reactions. More specifically, this invention relates to systems for and methods of characterizing parallel reactions on a chip.

### BACKGROUND OF THE INVENTION

Information related to the underlying mechanisms of biological function is increasing at an unprecedented rate. Methods to rapidly decipher vast amounts of DNA sequences fueled the genomic revolution. Although the resulting explosion of genetic information served to answer many questions, a far greater number of questions were raised and the need to develop new approaches to even begin to address these new questions was revealed. Thus, the proteomic and other similar-omic revolutions were born. Likewise, newly sequenced genomes have been riddled with interpretive holes termed “hypothetical proteins” and the like. This has provided the driving force for efforts to rapidly crystallize and solve protein structures in the hope that function will be revealed where sequence information has failed to provide a complete picture.

In the midst of these developments, the ability to carry out comprehensive evaluation of the catalytic performance of enzymes and other kinetic aspects of protein function has lagged far behind. Catalysis is the defining feature of enzyme function, and kinetic analysis of the transformations mediated by proteins and enzymes is central to understanding and manipulating them and the biological processes of which they are a part. Consequently, there is a substantial and widening gap between enzyme sequential structural information on one hand, and a true understanding of the catalytic capabilities of these enzymes on the other. This disparity is aggravated by the fact that the catalytic performance of enzymes often displays a complex dependence on multiple factors. The procedures themselves are lengthy and laborious, prompting many to characterize enzyme catalysis with as few assays as possible. The danger is that the resulting low-resolution kinetic description will contain large gaps and potentially misleading trends.

Enzymes are proteins that catalyze chemical reactions. In enzymatic reactions, enzymes assist in converting starting materials or starting molecules, referred to as substrates, into different materials or different molecules, referred to as the products. Enzymes are required for assisting biological processes that need to proceed at high rates. Enzymes typically accelerate these biological processes in a catalytic fashion by lowering the activation energy in the reaction pathway between the substrates and the products. Many biological processes occur at rates that are millions of times faster in the presence of an enzyme than without the presence of the enzyme.

Kinetic activity of an enzyme can be affected by a number of factors, such as substrate concentration, temperature, pH, and inhibitor concentration, to name a few. Using prior art methods to fully characterize the kinetic activity or kinetic landscape of an enzyme under a variety of conditions is extremely laborious.

### SUMMARY OF THE INVENTION

The present invention is directed to a system and device for and a method of characterizing reactions over a wide range of conditions using parallel reaction and detection techniques. Reagents used are in a gaseous state, a liquid state or a combination thereof. Reagents include but are not limited to biological reagents, such as bacterial, fungal, viral and richechia biological reagents.

The present invention is used to characterize binding reactions, combinatorial reactions enzymatic reaction, or any other reaction. In a particular embodiment of the invention the system and method of the present invention is used to characterize kinetic activities of catalysts, such as an enzymes. Finally, it is clear that devices based on those here could easily be applied to other biokinetic problems like protein folding/unfolding, protein:protein association, binding kinetics, and protein:nucleic acid association.

A system of the present invention includes an optical unit. In accordance with the embodiments of the invention the optical unit is an optical microfluidic unit. The optical microfluidic unit includes a microfluidic controller chip with multiple reaction cells, inlet ports and outlet ports. The microfluidic controller chip can be formed from two or more layers, as described below.

The reaction cells, inlet ports and outlet ports can have any suitable arrangement or architecture on the microfluidic controller chip. For example, the inlet ports and outlet ports are arranged on or along the periphery of the microfluidic controller chip, wherein the reaction cells are surrounded by the inlet ports and outlet ports. Alternatively, reaction cells are arranged on or along the periphery of the microfluidic controller chip, wherein the inlet ports and the outlet ports are surrounded by the reaction cells.

The reaction cells can be arranged in a parallel architecture, with two or more rows of reaction cells, a circular architecture or any other suitable geometric or random arrangement that is suitable for the application at hand. In a particular embodiment of the invention, the microfluidic controller chip is circular or disc-shaped with the reaction cells arranged in a circular-fashion or architecture on or along the periphery of the microfluidic controller chip and with the inlet ports and outlet ports being surrounded by the reaction cells. Regardless of the shape of the microfluidic controller chip or the particular arrangement or architecture of the inlet ports, outlet ports and reaction cells, the reaction cells themselves are preferably rotary reaction cells configured to hold nanoliter volumes or less of the reagents.

The system or optical microfluidic unit of the present invention preferably includes a detection unit for simultaneously monitoring concentrations of one or more reagents and/or products within each of the reactor cells. The detection unit includes one or more of an optical detector, an electrochemical detector and a mass-based cantilever detector. Where the detection unit includes optical detector unit, The optical detector unit preferably includes a light source, such as an array of light emitting diodes and a detector, such as a photodiode array. The photodiode array can be a charge-coupled diode array (CCD), an avalanche photodiode array (APD) or a CMOS integrated p-n diode array. The light source and the detector preferably sandwich the microfluidic controller chip, such that the optical detection means simultaneously monitors concentrations of one or more of the reagents and/or products within each of the reaction cells by detecting light from the source that passes through the microfluidic controller chip and determining absorbance values for each of the reaction cells.

In accordance with further embodiments, the system or optical microfluidic unit includes a temperature controller. The temperature controller is for controlling temperatures of the microfluidic controller chip or the reaction cells of the microfluidic controller chip by one or more of thermal contact and optical heating. Materials and methods for making temperature controllers are further described in the U.S. Provisional Patent Application Ser. No. 60/798,604, titled “MICROFLUIDIC CHIP FOR PROTEIN KINETICS,” and the U.S. Provisional Patent Application Ser. No. 60/843,385, titled “REACTION KINETIC LANDSCAPER,” referenced previously.

The system of the present invention also includes an actuator device coupled to the microfluidic controller chip. In accordance with the embodiments of the invention, the actuator device is a microfluidic pump that is coupled to the microfluidic controller chip through the inlet ports using any suitable plumbing or piping. The microfluidic pump is configured to inject and mix samples of the catalyst with reagents within the reaction cells to form products. Reagents include, but are not limited to, buffers, solvents, biological substrates and enzyme inhibitors.

The system of the present invention is preferably automated and computerized. In accordance with the embodiments, a computer includes a processor and memory. The computer is programmed with the software that interfaces with the microfluidic pump, optical detection means and the temperature controller, such that the computer controls reaction conditions, collects optical data and stores the optical data acquired by the optical detection means. Preferably, the computer includes software to calculate kinetic parameters of the catalyst being studied from the optical data acquired through multiple runs of a number of parallel or simultaneously monitored reactions, such as described above. The computer is also preferably configured to use the kinetic parameters to plot a graphical “landscape” representation of the kinetic activity of the catalyst. For example, the computer is configured to plot a contour surface of the kinetic parameters, which is displayed on a display monitor or graphical user interface.

### BRIEF DESCRIPTION OF THE DRAWINGS

FIG. 1 is a schematic diagram of an automated computerized system with an optical microfluidic unit for characterizing kinetic activity of a catalyst, in accordance with the embodiments of the invention.

FIG. 2 is a schematic diagram of an optical microfluidic unit for characterizing a kinetic activity of a catalyst, in accordance with the embodiments of the invention

FIG. 3 is a detailed schematic diagram of an automated computerized system with an optical microfluidic unit, a light source and detector for optically characterizing a kinetic activity of a catalyst, in accordance with the embodiments of the invention.

FIG. 4A is a diagram of a microfluidic controller chip with inlet ports, outlet ports, and rotary reaction cells, in accordance with the embodiments of the invention.

FIG. 4B is a diagram of a circular or disc-shaped microfluidic controller chip with inlet ports and outlet ports surrounded by a circular arrangement or architecture of rotary reaction cells, in accordance with the embodiments of the invention.

FIG. 5 is a graph of a contour surface that characterizes a landscape of kinetic activity for a catalyst, in accordance with the embodiments of the invention.

FIG. 6A is a graphical representation of the steps to generate a contour surface for characterizing a landscape of kinetic activity for a catalyst, in accordance with the embodiments of the invention.

FIG. 6B is a block-flow diagram outlining the steps for characterizing a landscape of kinetic activity for a catalyst, in accordance with the embodiments of the invention.

FIG. 7A outlines processing options for data collected from a single sector of the 48-channel control chip, in accordance with the embodiments of the invention.

FIG. 7B is a flow-chart for instantaneous calculation of Vₘₐₓ, K_{M} and k_{cat} using rate data from multiple, parallel enzyme reactions, in accordance with the embodiments of the invention.

FIGS. 8-23 illustrate an embodiment of a controller chip design including four layers.

FIGS. 24-36 illustrate an embodiment of a controller chip design including two layers.

### DETAILED DESCRIPTION OF THE INVENTION

An enzyme (E) binds a substrate (S) and produces a product (P). The kinetic properties of an enzyme can be described by Michaelis-Menten kinetics. Michaelis-Menten kinetics are derived from the premise that a substrate binds reversibly to the enzyme, forming the enzyme-substrate complex. The enzyme then catalyzes the chemical step in the reaction and releases the product.

Saturation curves for an enzyme reaction are able to be generated to show a relationship between the substrate concentration (S) and the rate (V). The rate (V) at which the enzyme catalyzed reaction occurs depends on a number of factors including, but not limited to, solution conditions and substrate concentration.

To determine a substrate concentration where the rate (V) at which the enzyme catalyzed reaction is greatest (Vmax), the substrate concentration is increased until a constant rate of product formation is observed. The rate Vmax or saturation occurs when all or most of the enzyme is complexed with the substrate (ES).

From the data collected to determine the rate Vmax, the Michaelis-Menten constant (Km) is also able to be determined. The Michaelis-Menten constant (Km) is equal to one-half Vmax. Each enzyme has a characteristic Km for a given substrate. Accordingly, the characteristic Km is often used to characterize binding properties of the substrate.

Another constant that can be determined from the data collected to determine the Vmax is the constant kcat, which is the number of substrate molecules handled by one active site per second. The efficiency of an enzyme is able to be expressed in terms of kcat/Km, also called the “specificity constant.”

Regardless of what simple or complex kinetic model is used to analyze the kinetic data of an enzyme, the kinetic data is obtained through assays that are laboriously performed using manual micro-pipet techniques. A system for and method of collecting large quantities of kinetic data for catalysts, such as enzymes, using parallel and automated processing of microfluidic reactions and data collected therefrom is herein described. It will be clear to one skilled in the art that the system and method of the present invention is also able to be used to monitor and characterize any number of reactions, including but not limited to binding reactions, combinatorial reactions and enzymatic reactions. Reagents used are in a gaseous state, a liquid state or a combination thereof. Reagents include but are not limited to biological reagents, such as bacterial, fungal, viral and richechia biological reagents. The present invention is envisioned to have applications in the study of mammalian cells.

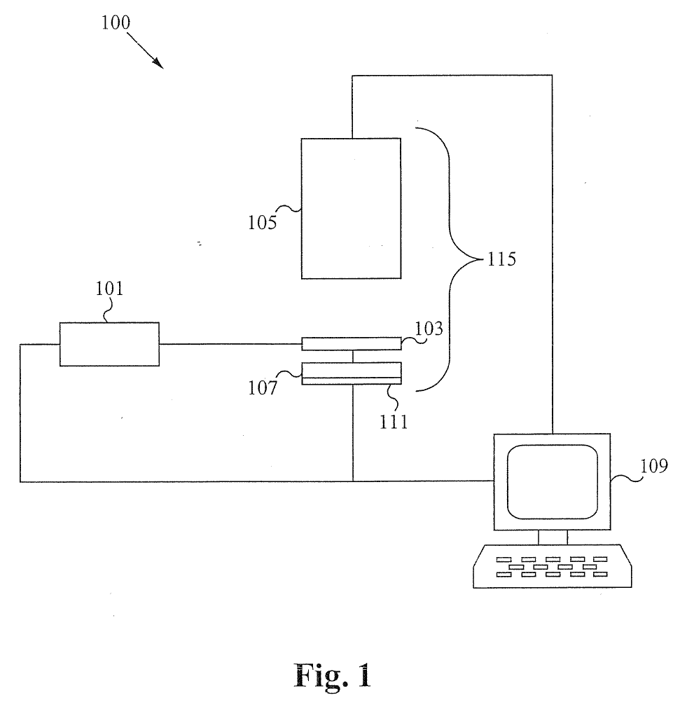

FIG. 1 is a schematic diagram of an automated computerized system 100 with an optical unit 115 for characterizing kinetic activity of a catalyst. In accordance with the embodiments of the invention the optical unit 115 is an optical microfluidic unit. The optical microfluidic unit 115 includes a controller chip 103 with multiple reaction cells, inlet ports and outlet ports, such as described below. The optical microfluidic unit 115 also includes an optical detection means 105 and 111. In accordance with the embodiments, the optical detection means 105 and 111 includes a light source 105 and a detector 111.

Still referring to FIG. 1, the system 100 also includes a microfluidic pump 101 coupled to the controller chip 103 and a thermal controller 107. The microfluidic pump 101 is configured to inject and mix samples of the catalyst with reagents within the reaction cells through actuation lines to form products. Reagents include, but are not limited to, buffers, solvents, biological substrates and enzyme inhibitors. The microfluidic pump 101 is coupled to the controller chip 103 through the inlet ports using any suitable plumbing or piping. Preferably, each actuation line is coupled to the controller chip 103 through a stainless steel pin and polyethylene tubing. The microfluidic pump 101 includes solenoid valves controlled by a digital data I/O card (not shown). It will be clear to one skilled in the art that the microfluidic pump 101 is not required and the mixing is able to alternatively be controlled using capillary forces or any other suitable mechanism inherent to the controller chip 103 or external to the controller chip 103.

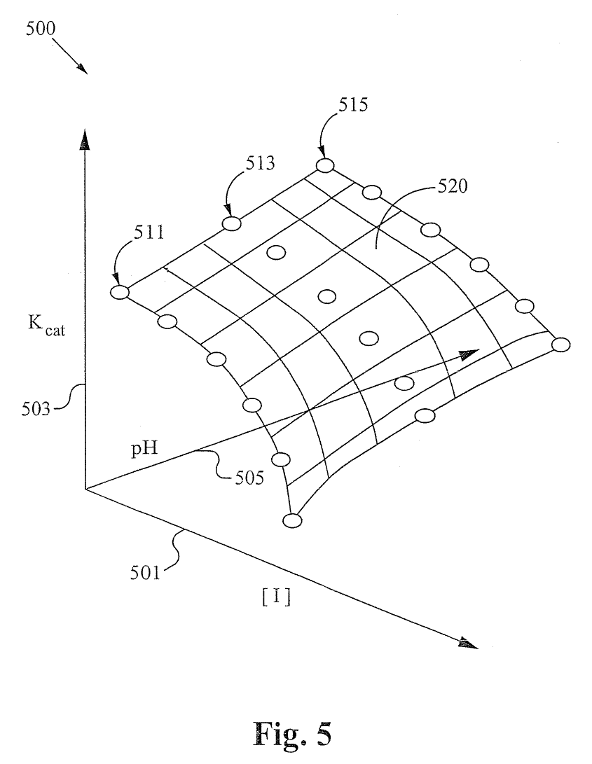

The system 100 also preferably includes a computer 109 with a processor and memory. The computer 109 is in communication with the optical microfluidic unit 115 and the microfluidic pump 101. The computer 109 preferably includes software to calculate kinetic parameters of the catalyst being studied from the optical data acquired from the optical detection means 105 and 111. The computer 109 is also preferably configured to use the kinetic parameters to plot a graphical “landscape” representation of the kinetic activity of the catalyst, such as a contour surface 520 shown in FIG. 5.

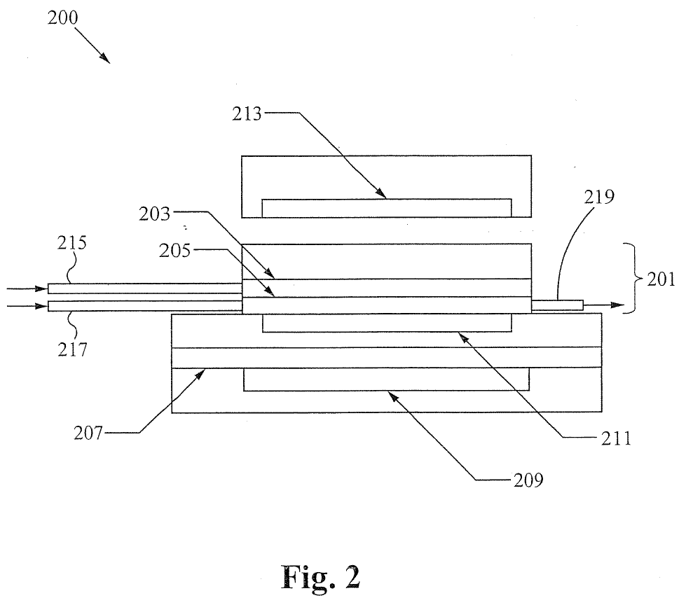

FIG. 2 shows an optical microfluidic unit 200, similar to the optical microfluidic unit 115 described above with reference to FIG. 1. Throughout this specification, identically labeled elements refer to the same element. The optical microfluidic unit 200 includes a controller chip 201. The controller chip 201 is formed from at least two layers, such as a control layer 203 and a microfluidic layer 205. The layers 203 and 205 are coupled to a microfluidic pump 101 (FIG. 1) through actuation lines 215 and 217 that are coupled to the controller chip 201 through inlet ports, such as described below. The layers 203 and 205 are also coupled to one or more drainage lines 219 that connect to the controller chip 201 through one or more corresponding outlet ports, also described below.

The optical microfluidic unit 200 includes an optical detection means that includes a light emitting diode array 213 and a photodiode array 209. The optical detection means also preferably includes a suitable insulation and/or optical filtering layer 207. Preferably, the light emitting diode array 213 and the photodiode array 209 sandwich the controller chip 201, such that the optical detection means monitors and determines absorbance values for each reaction cell of the controller chip 201.

Still referring to FIG. 2, the optical microfluidic unit 200 also preferably includes a thermal controller 211, that is in thermal contact with the controller chip 201. The thermal controller 211 is designed to maintain constant and consistent temperatures with each of the reaction cells of the controller chip 201 over the duration of the reaction times. The thermal controller 211 is preferably transparent or substantially transparent to the light generated by the light emitting diode array 213. The thermal controller 211 is formed from any suitable material, including transparent metal layers or metal oxide layers deposited on patterned or unpatterned glass.

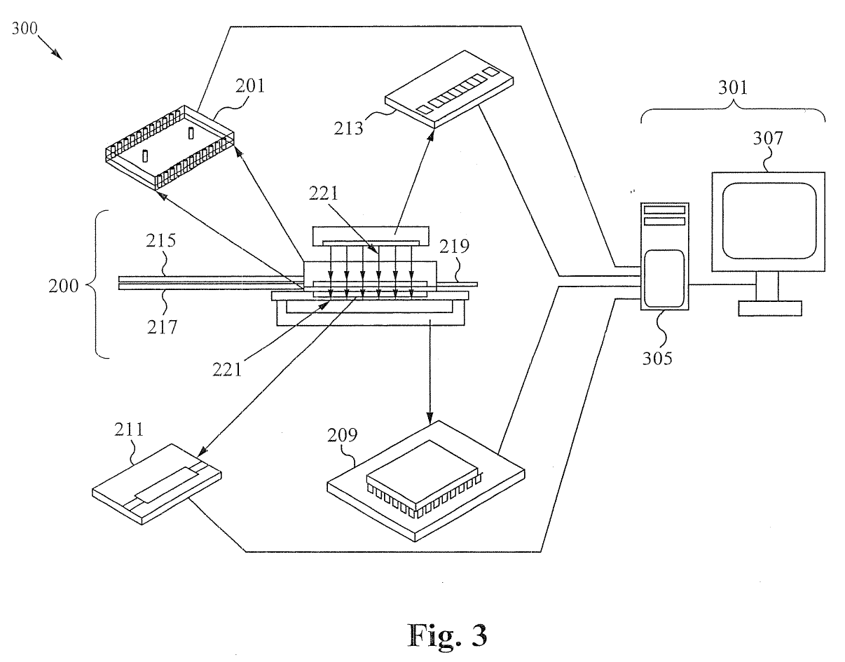

FIG. 3 is a detailed schematic diagram of an automated computerized system 300 with the optical microfluidic unit 200, described above. As shown, the system 300 includes a computer 301 with a processor 305 and a monitor 307. Preferably, the computer 301 is integrated with the controller chip 201, light emitting diode array 213, photodiode array 209 and the thermal controller 211, such that the computer 301 is able to control and monitor the entire operation of the optical microfluidic unit 200 and the data acquisition performed thereon. The system 300 also includes a microfluidic pump (not shown), such as the microfluidic pump 101 (FIG. 1) that is coupled to the optical microfluidic unit 200 through the actuation lines 215 and 217. The light emitting diode array 213 and the photodiode array 209 sandwich the controller chip 201, such that the optical detection means monitors and determines absorbance values for each reaction cell of the controller chip 201 from an amount of light that passes through the controller chip 201, as indicated by the arrows 221.

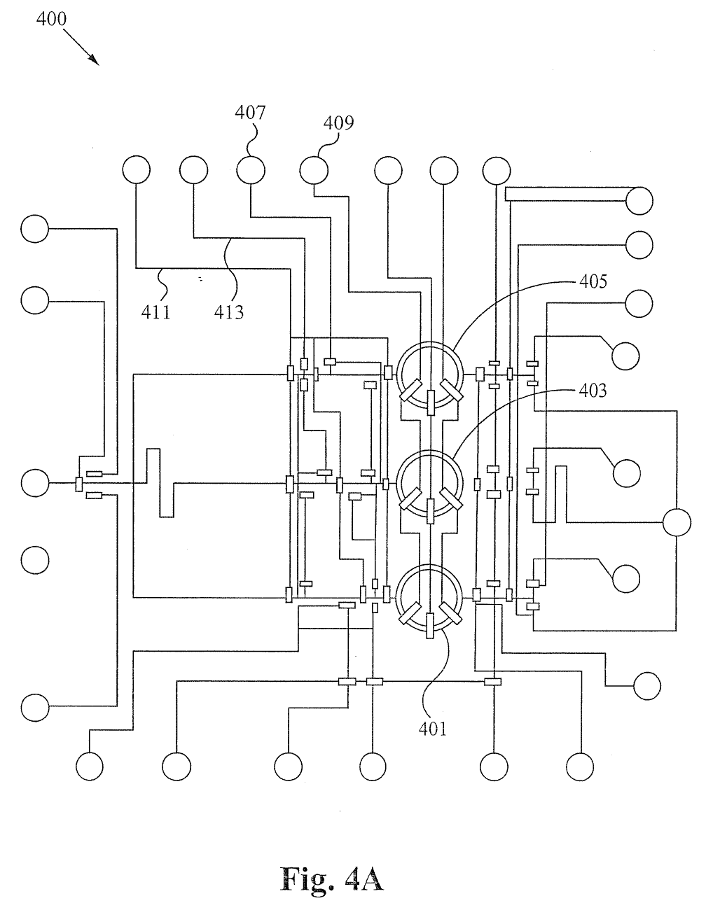

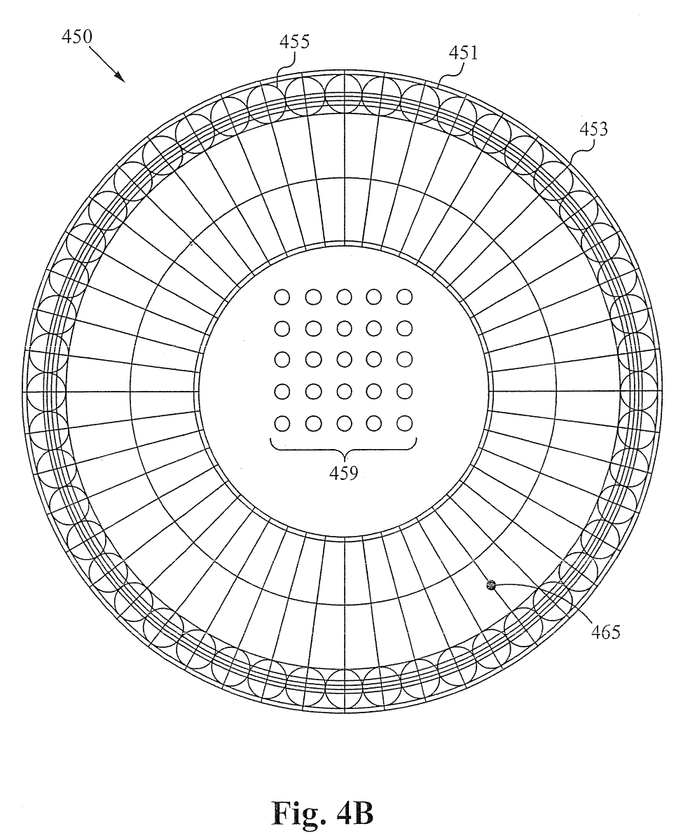

FIG. 4A is a diagram of a controller chip 400 with ports 407 and 409 for coupling to actuation lines and drainage lines, such as described above. The controller chip 400 includes multiple reaction cells 401, 403 and 405 that are used for simultaneously reacting samples of a catalyst with a range of reaction conditions and simultaneously monitoring each of the multiple reaction cells 401, 403 and 405 to characterize the kinetic properties of the catalyst. The reaction cells 403 and 405 are preferably rotary reaction cells, such as shown, that are coupled to the ports 407 and 409 through any number of channels 411 and 413.

FIG. 4B is a diagram of a circular or disc-shaped controller chip 450 with inlet ports and outlet ports 459 surrounded by a circular arrangement or architecture of rotary reaction cells 451, 453 and 455. The circular or disc-shaped controller chip 450 is configured with channels 465 for injecting samples of enzymes and reagents into each of the rotary reaction cells 451, 453 and 455, such as described above.

FIG. 5 shows a graphical representation 500 of a contour surface 520 generated from kinetic data acquired in accordance with a method of the invention. The axis 501 represents inhibitor concentration (I), the axis 503 represents the number of substrate molecules handled by one active site per second (kcat) and the axis 505 represents pH. The reaction sequences 511, 513 and 515 are shown as a range of three pH values.

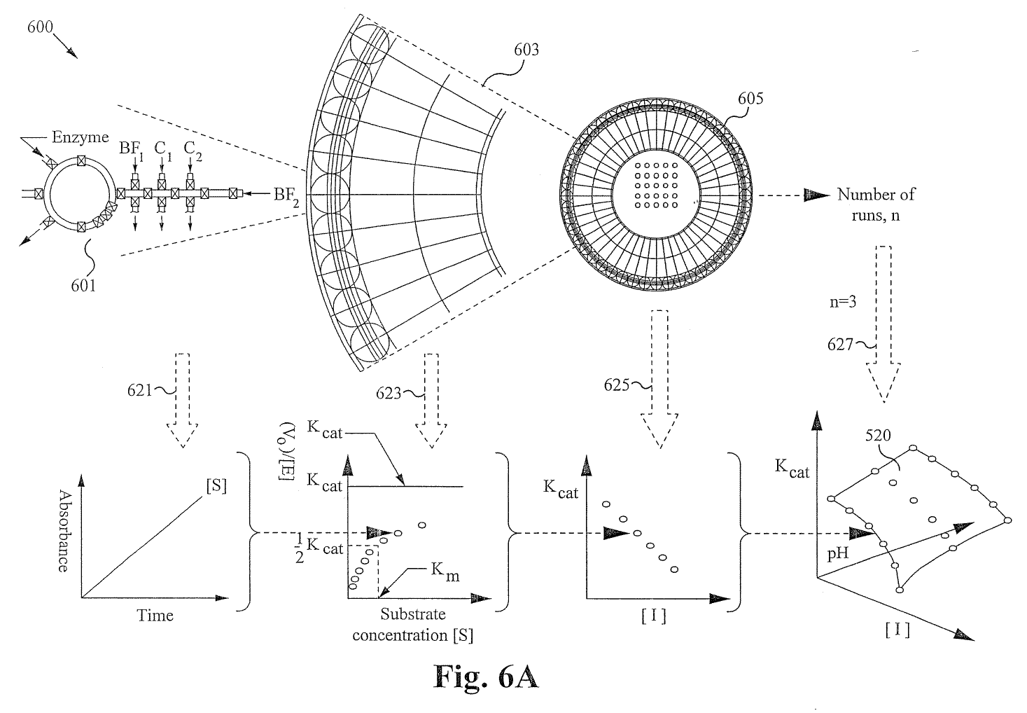

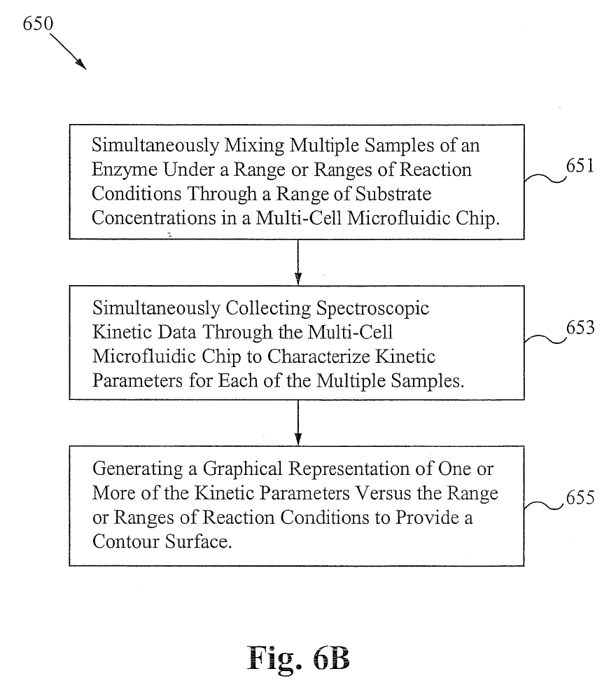

FIG. 6A is a graphical representation 600 of the steps to generate the contour surface 520 (FIG. 5) for characterizing a landscape kinetic activity for a catalyst, in accordance with the embodiments of the invention. In the step 601, a sample of an enzyme is reacted with a substrate in a rotary cell controller chip 605 at a selected pH and a selected inhibitor concentration. In the step 621, the reaction is monitored by measuring optical absorbance, such as described above. The reaction can be monitored by measuring optical absorbance of the substrate, a product formed by the reaction or a combination thereof.

Still referring to FIG. 6A, in the step 623, the reaction is carried out within the multiple cells 603 of the controller chip 605 at the selected pH value and inhibitor concentration over a range of substrate concentrations (S) to derive kinetic parameters. In the step 625, the steps of 621 and 623 are carried over a range of inhibitor concentrations. The steps 621, 623, and 625 are then repeated a number of times (n) over a range of pH values to generate the contour surface 520 in the step 627.

FIG. 6B is a block-flow diagram 650 outlining the steps for characterizing a landscape for kinetic activity of a catalyst, in accordance with a preferred method. In the step 651, multiple reactions are simultaneously carried out in multiple cells of a controller chip over a range of reaction conditions. For example, multiple reactions are able to be carried out over a range of substrate concentrations at a constant pH value and inhibitor concentration. In the step 653 all of the reaction cells are simultaneously monitored using spectroscopic techniques to obtain kinetic parameters at the range of reaction conditions. After the kinetic parameters are obtained in the step 653, in the step 655 a graphical representation of one or more of the kinetic parameters versus the range reaction conditions (i.e. substrate concentrations) is generated, similar to that described with reference to step 623 in FIG. 6A.

Still referring to FIG. 6B, in further embodiments, the steps 651 and 653 are repeated at multiple reaction conditions. For example, multiple reactions are carried out over a first set of reaction conditions, within a range of substrate concentrations at a constant pH value and inhibitor concentration. In the step 653, all of the reaction cells processed at the first set of reaction conditions are simultaneously monitored using spectroscopic techniques to obtain kinetic parameters within the range of reaction conditions. After the kinetic parameters are obtained in the step 653, in the step 651 multiple reactions are carried out over a second set of reaction conditions, wherein a range of inhibitor concentrations at a constant pH and substrate concentration. In the step 653, all of the reaction cells processed at the second set of reaction conditions are simultaneously monitored using spectroscopic techniques to obtain kinetic parameters within the range of reaction conditions. After the kinetic parameters are obtained in the step 653 from the first and second set of reaction conditions, in the step 655 a graphical representation of one or more of the kinetic parameters versus the range of reaction conditions (i.e. substrate concentrations and inhibitor concentrations) is generated, similar to that described with reference to step 625 in FIG. 6A. It will be clear to one skilled in the art that the procedure described above is able to be repeated any number of times to provide a graphical landscape representation of the kinetic characteristics of a catalyst being studied.

Still referring to FIG. 6B, in yet further embodiments, several sets of reactions are simultaneously processed and monitored using sections of the controller chip 605. For example, in the step 651 multiple reactions are carried out over a first set of reaction conditions in a first section of the controller chip 605, simultaneously multiple reactions are carried out over a second set of reaction conditions in a second section of the controller chip 605, and simultaneously multiple reactions are carried out over a third set of reaction conditions in a third section of the controller chip 605. In the step 653, all of the reaction cells processed at the first second and third sets of reaction conditions in the first, second and third section of the controller chip 605 are all simultaneously monitored using spectroscopic techniques to obtain kinetic parameters within the range of reaction conditions. After the kinetic parameters are obtained in the step 653 from the first and second sets of reaction conditions, in the step 655 a graphical representation of one or more of the kinetic parameters versus the range of reaction conditions is generated, similar to that described with reference to step 627 in FIG. 6A.

The design of the microfluidic chip ensures the rapid, parallel collection of reaction data for multiple enzyme reactions. Therefore, the tools to equally rapidly process, analyze, and plot these data are necessary. To address this gap, easy-to-use software to process the collected enzymatic reaction data and return a comprehensive plot will be developed. Software for enzyme kinetic analyses must recast enzyme reaction data in a form amenable to rapid and accurate plotting. Software has been developed to control and visualize microfluidic chip operation and data processing for microfluidic applications, with the user-friendly visual programming language, LabView.

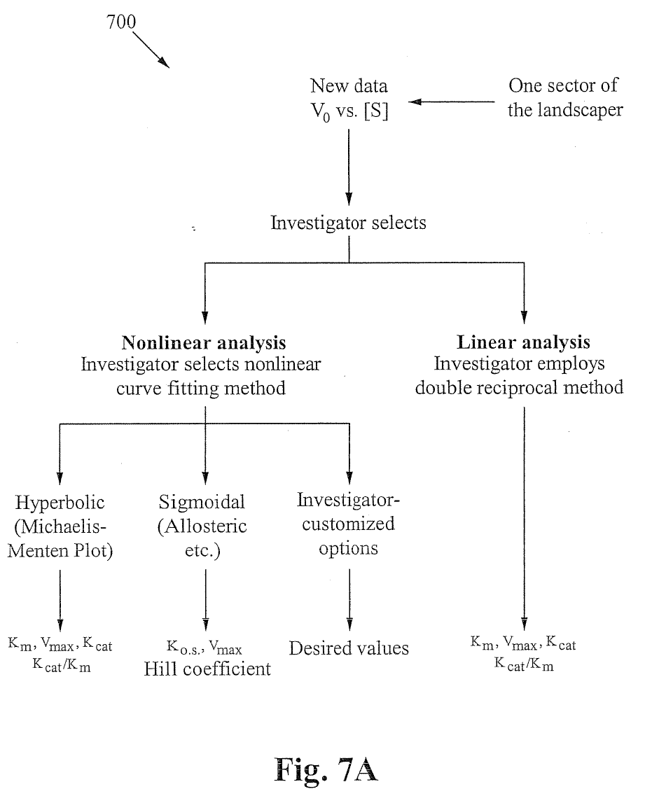

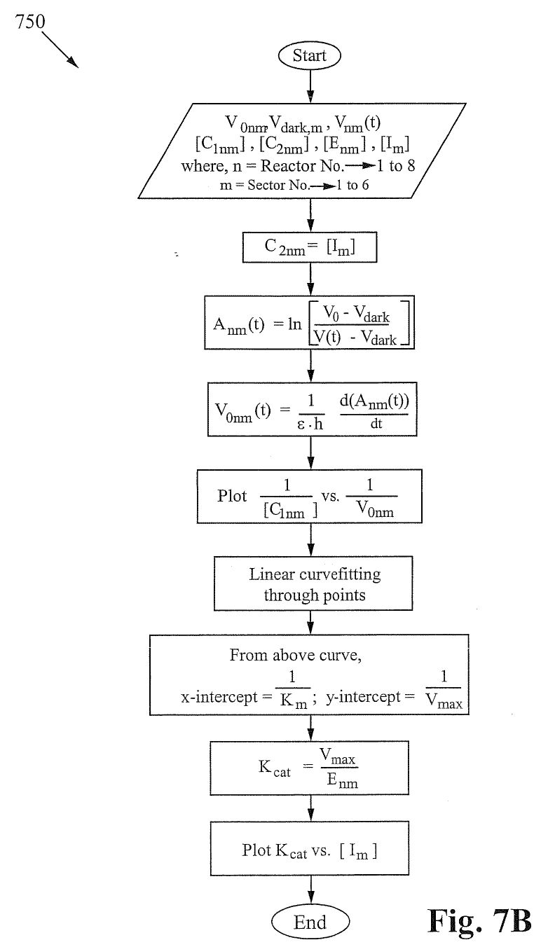

FIG. 7A outlines processing flow options 700 for data collected from a single sector of the 48-channel control chip, in accordance with the embodiments described herein. The data from a single sector of the 48-channel chip (or cell control chip; FIG. 4B) obtained through a program, operating on a system of the invention, is used to generate plots of vₒ versus [S] in non-linear or linear (e.g., double-reciprocal) format. The plot will instantly return the values of the enzyme kinetic parameters such as K_{M} and Vₘₐₓ for the reaction conditions corresponding to that sector. As shown in FIG. 7A, the design of the system will allow an investigator considerable freedom in data analysis. Either linear (e.g., double reciprocal) or nonlinear fitting routines are available to the investigator. In the event that a particular enzyme being studied exhibits a kinetic behavior that does not follow Michaelis-Menten kinetics, other nonlinear fitting routines will be available and the investigator will have the option of inputting his/her own customized equations for nonlinear analysis.

FIG. 7B shows a flow-chart 750 for instantaneous calculation of the values Vₘₐₓ, K_{M} and k_{cat}, using rate data from multiple, parallel enzyme reactions, in accordance with the embodiments of the invention. As shown in the flow-chart 750, conversion of raw kinetic data collected by the system of the invention is converted to the kinetic parameters Vₘₐₓ, K_{M} and K_{cat}. The photodiode voltage corresponding to zero absorbance is V₀ (not to be confused with initial rate [Vₒ]), the value V_{dark} is the photodiode dark voltage, and the value V(t) is the photodiode voltage with respect to time. Of course, the biokinetic landscaping chip allows the independent variation of two components (C₁ and C₂) where a component is a substrate, inhibitor, or other factor.

The optical detection system works on the principle of absorption spectroscopy or spectrophotometry. In a spectrophotometer, light absorption of a sample (in the case of enzyme kinetics, absorption of enzyme product) is able to be related to the concentration of that sample. Of course, this relation is described by the well-known Beer-Lambert law.
A(t)A_{blank}=εbc

Here, the value A is the unitless absorbance of the sample at some wavelength. The term ε refers to the extinction coefficient (or millimolar absorptivity) of the chromophore (mM⁻¹ μm⁻¹), the value c is the concentration (mM) of the sample, and the value b is the path length of the sample (in our case the height of microfluidic channel in μm).

In the case of integrated photodiode-based optical detection systems, absorbance is given in terms of voltage from the photodiode. The absorbance at time t of enzyme product, A(t), is proportional to voltages of the photodiode and is related by the equation below.

$$A ( t ) = ln \frac{{I}_{0}}{I ( t )} = ln \frac{ {V}_{0} - {V}_{dark}}{V ( t ) - {V}_{dark}}$$

Where the value lₒ is intensity of light corresponding to zero absorbance, the value l(t) is intensity of light related to absorbance with related to time, the value V(t) is voltage of the photodiode corresponding to change in absorbance with related to time the value Vₒ is voltage of photodiode corresponding to zero absorbance, the value V_{dark} is voltage of the photodiode in dark conditions. The velocity of enzyme product formation is a function of absorbance, extinction coefficient and height of the microfluidic channel and can be derived as follows.

$${V}_{0 nm} ( t ) = \frac{1}{ɛ h} \frac{ⅆ \{ {A}_{nm} ( t ) \}}{ⅆ t}$$

By making use of calculated velocity and substrate concentration, the investigator is free to plot the data in linear or nonlinear formats as desired. The appropriate kinetic constants are returned and can be applied to the kinetic landscape for further analysis. Strictly speaking, maximum observed rates will be returned as enzyme concentration-dependent terms (e.g., Vₘₐₓ). Part of the program set-up will include a field for entry of the known enzyme concentration used for the kinetic experiments. In this way, the corresponding enzyme-concentration independent parameters (e.g., K_{cat} or turnover number) will also be calculated during data analysis by the software.

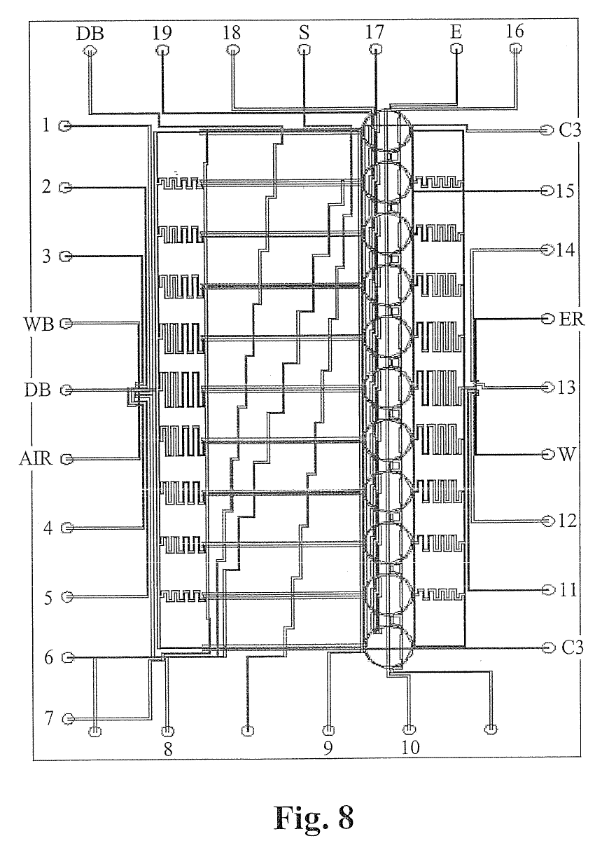

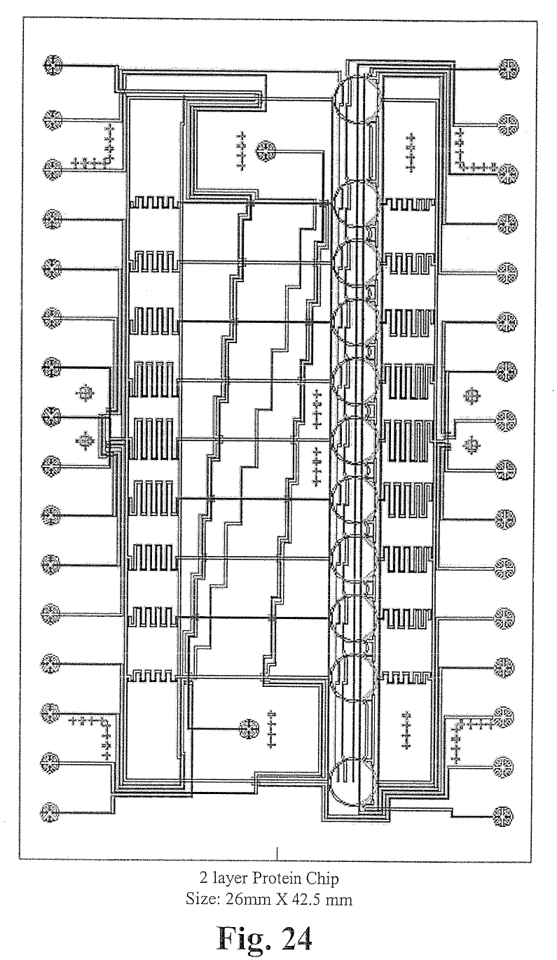

FIGS. 8-23 are used to illustrate a controller chip design including four layers and FIGS. 24-36 are used to illustrate a controller chip design including two layers. It will be clear to one skilled in the art that from the discussion above and the discussion below that the controller chip of the present invention can have any number of different designs or architectures, including any appropriate number of layers.

FIG. 8 illustrates an embodiment of the chip design including four layers. This design is realized in four layers from any kind of flexible material such as PDMS. Out of the four layers, two layers are thick slabs and two layers are thin films. The thick slabs are used for fluidic sample flows and the thin films are utilized for control layers. For better understanding, the numbering of the layers is started from the bottom to the top. In the four layer chip design, the first and the third layers are thin control layers, while the second and the fourth layers are thick fluidic layers. In addition, the four layer chip design consists of complicated and multiple parallel processors with different mixing ratios of reagents such as the dilution buffer (DB) and the substrate (S).

Within the chip design illustrated in FIG. 8, the first layer from the bottom is a control Layer 1, “C1.” The Control Layer 1 “C1” is used for control of the second layer, known as the fluidic processor layer “FPL.” The Fluidic Processor Layer “FPL” is intended for metering and mixing of the reagents in the parallel processors. Hence, the second layer is known as the fluidic processor layer FPL. The third layer is a Control Layer 3, “C3.” The Control Layer 3 “C3” is used for control of the fourth layer, known as the fluidic supply layer “FSL.” The Fluidic Supply Layer “FSL” is meant for supply of reagents to the parallel processors. Hence, the fourth layer is known as the fluidic supply layer FSL.

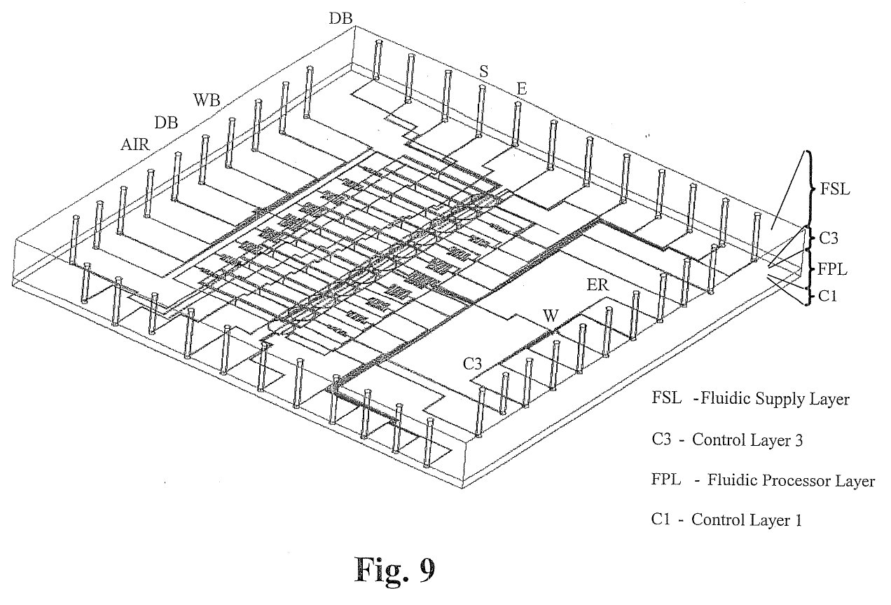

FIG. 9 illustrates a three dimensional (3D) view of the four layer chip design. These layers are explained above in relation to FIG. 8 and are shown on the three dimensional diagram in FIG. 9. The first control layer C1 from the bottom is in contact with any other clean and flat surface and is used for control of the second layer FPL. The second layer FPL is intended for metering and mixing of the reagents in the parallel processors and is called the fluidic processor layer FPL. The third control layer C3 is used for control of the fourth layer FSL. And finally the fourth layer is used for supply of reagents to the parallel processors and is known as the fluidic supply layer FSL, as discussed above.

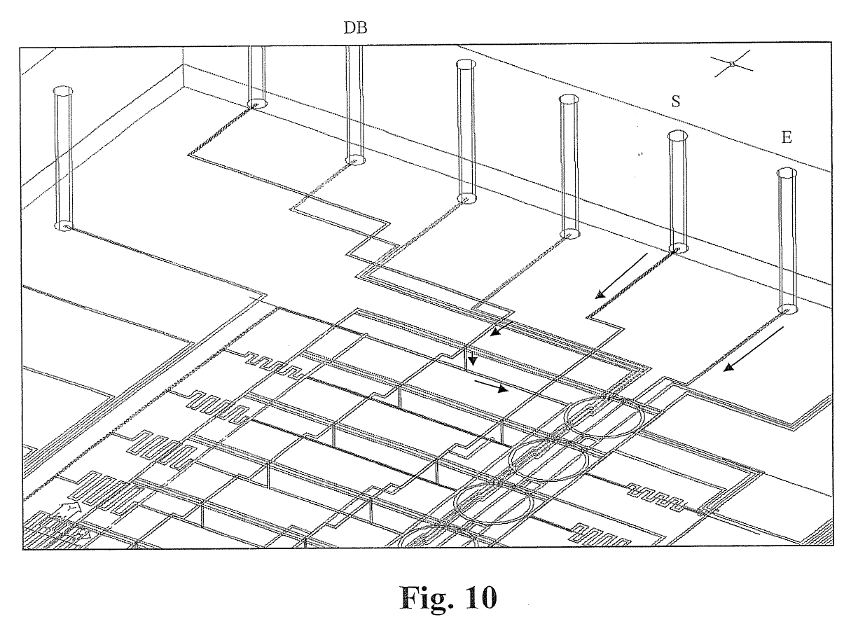

FIG. 10 illustrates a zoomed view of the four layer chip design. The arrows show direction of movement of reagents supplied from the fourth layer FSL channels to the second layer FPL parallel processors through the vertical round hollow holes.

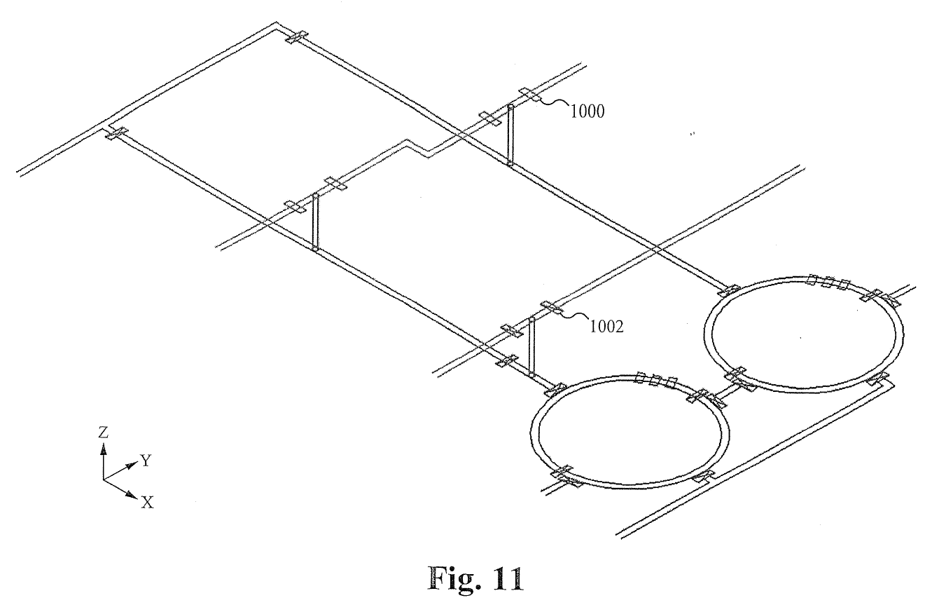

FIG. 11 illustrates the operation of the four layer chip design. To explain the operation of the four layer chip design, two processors having mixing ratios 10:0 and 9:1, respectively, are zoomed out. In the 10:0 processor, there is a 100% dilution buffer and 0% substrate. While in the 90:10 processor, there is a 90% dilution buffer and 10% substrate. In FIG. 11, the small hollow rectangles, such as 1000, indicate an open valve, while the crossed rectangles, such as 1002, indicate a closed valve. This designation of open and closed valves is used throughout the Figs. to be discussed below.

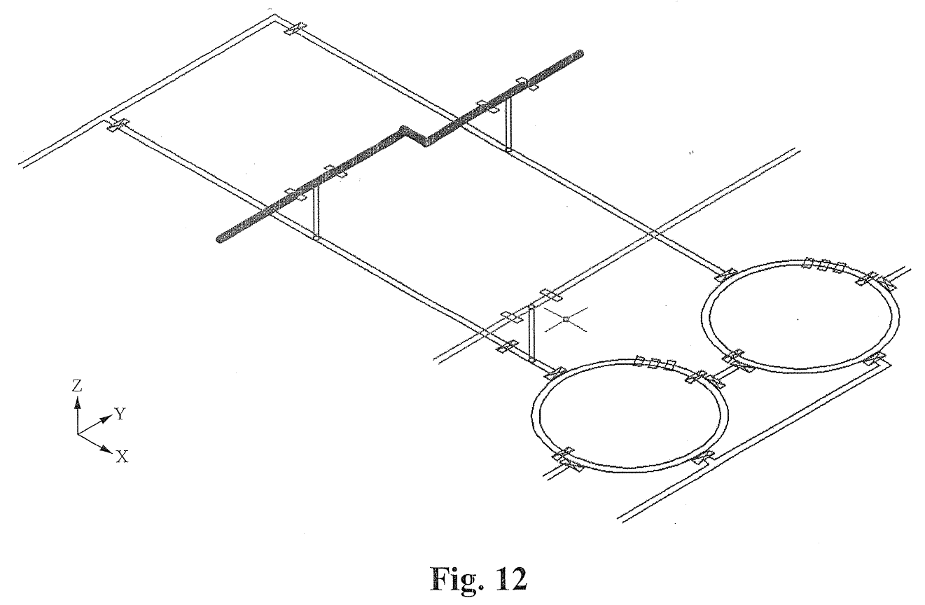

FIG. 12 illustrates opening of the valves in the third layer for the dilution buffer supply lines. Hence, the dilution buffer is spread into the fourth layer dilution buffer supply channel.

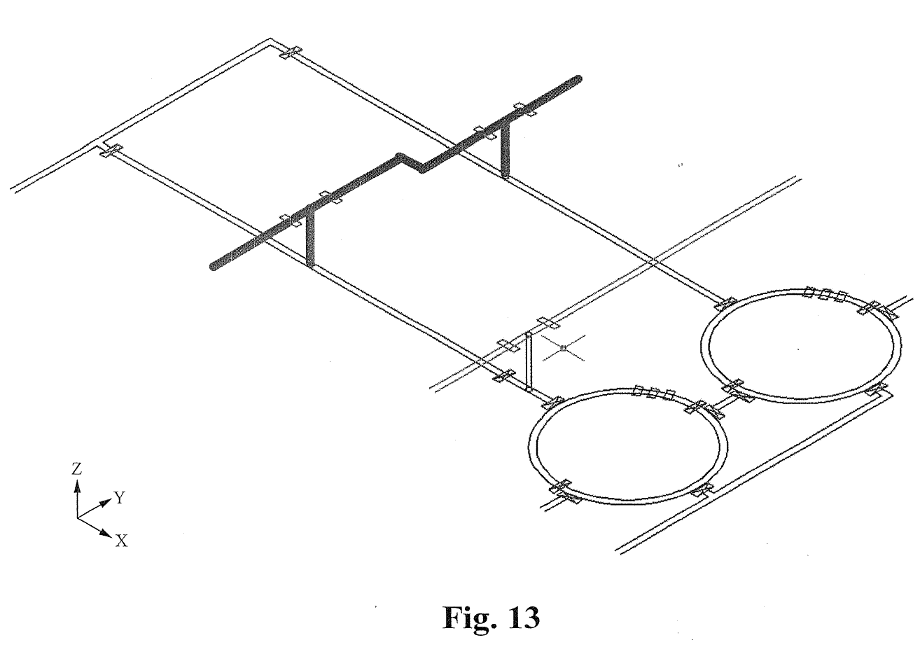

FIG. 13 illustrates the dilution buffer in the fourth layer supply channel descending down to the second layer through the vertical holes.

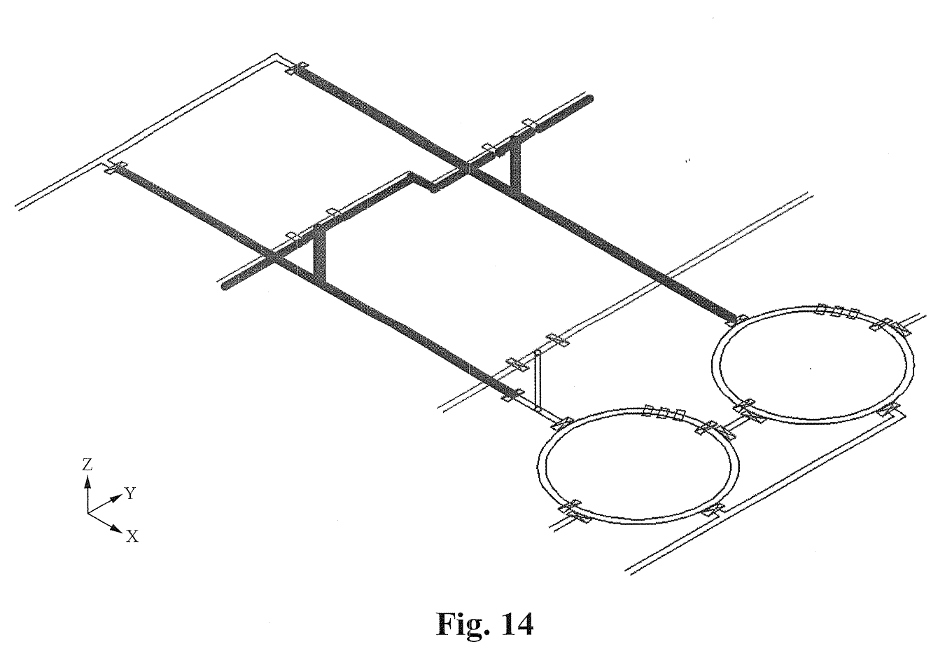

FIG. 14 illustrates the dilution buffer being spread in the second layer parallel processor dilution buffer metering channel. In the 10:0 processor, there is 100% dilution buffer. However in the 9:1 processor, there is 90% dilution buffer, with the remaining 10% currently empty and intended for substrate.

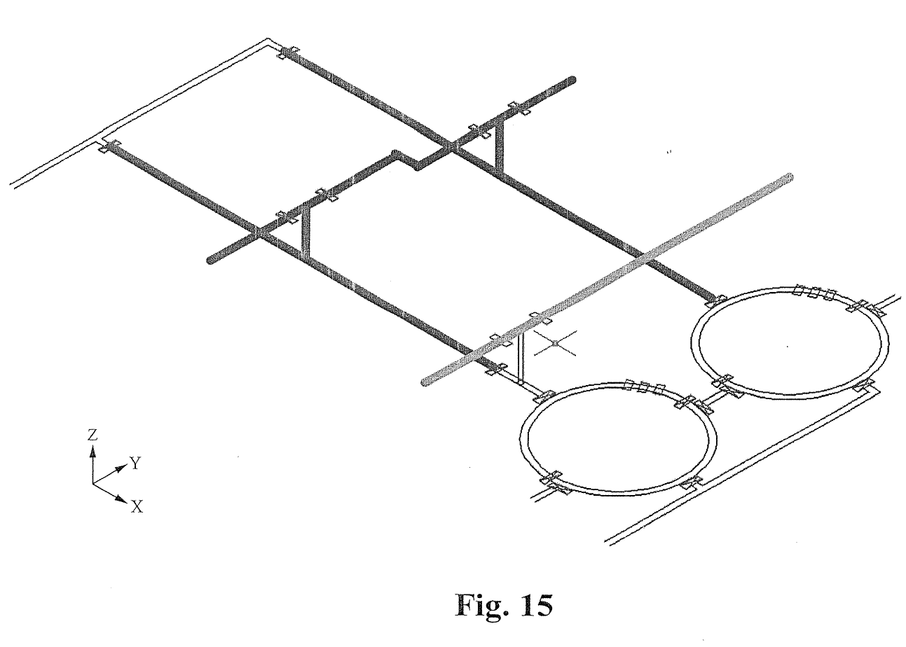

FIG. 15 illustrates the opening of the valves in the third layer for the substrate supply lines. Hence, the substrate is spread into the fourth layer substrate supply channel.

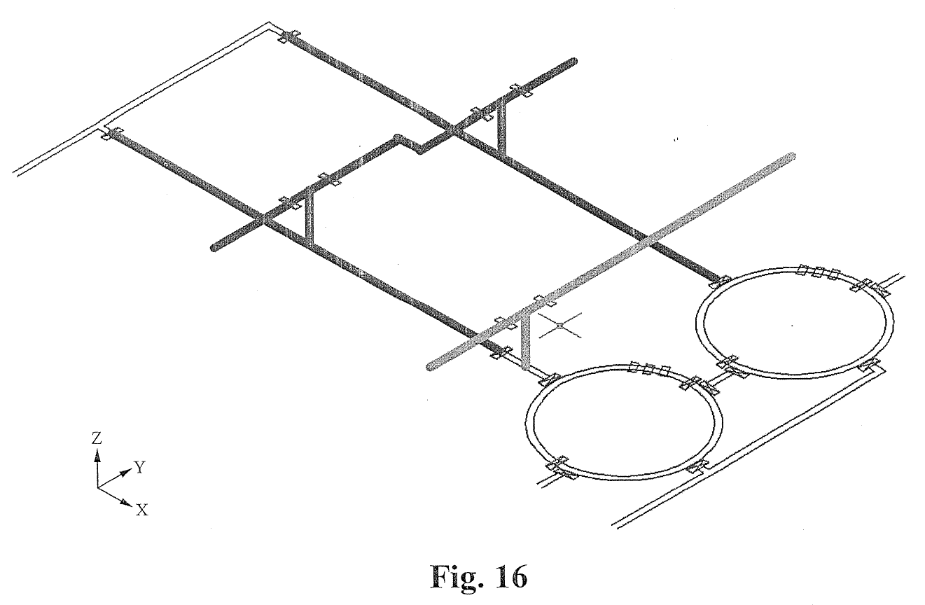

FIG. 16 illustrates the substrate in fourth layer supply channel descending down to the second layer through the vertical holes.

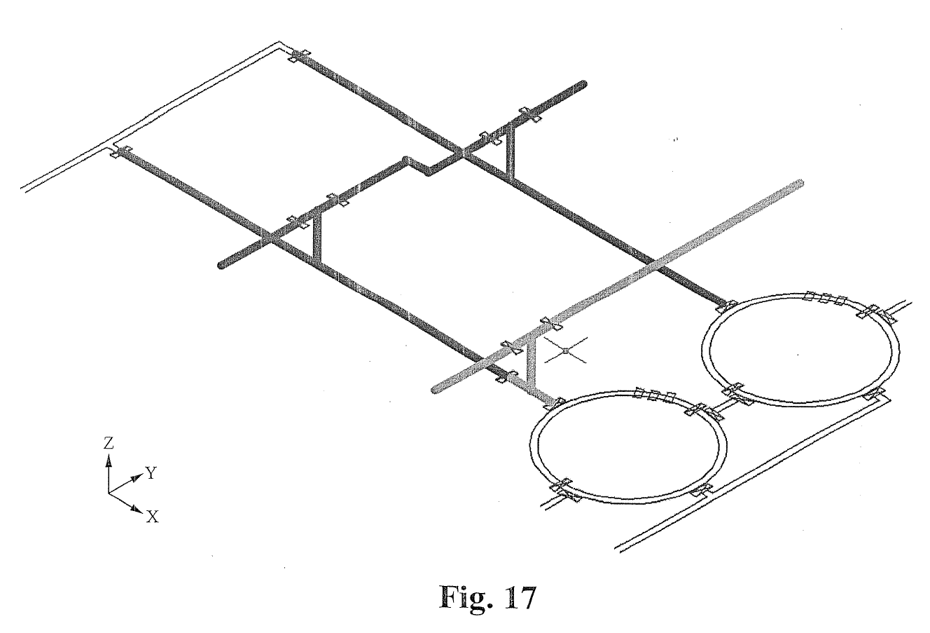

FIG. 17 illustrates the substrate being spread in the second layer parallel processor substrate metering channel. As illustrated in FIG. 17, in the 10:0 processor, there is 0% substrate. However in the 9:1 processor, there is 10% substrate.

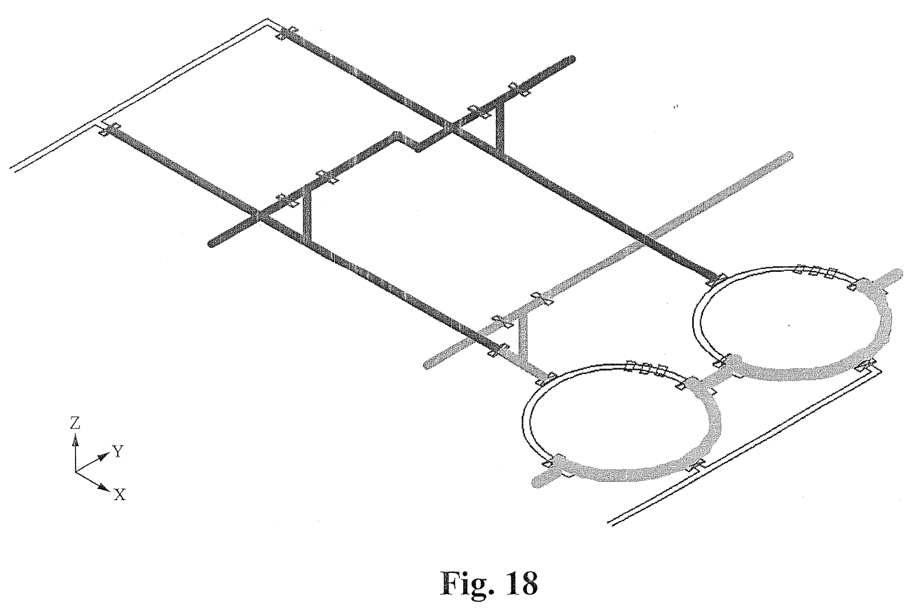

FIG. 18 illustrates the opening of the valves in the first layer for the enzyme supply lines. Hence, the enzyme is spread directly into the second layer enzyme portion in the mixing ring.

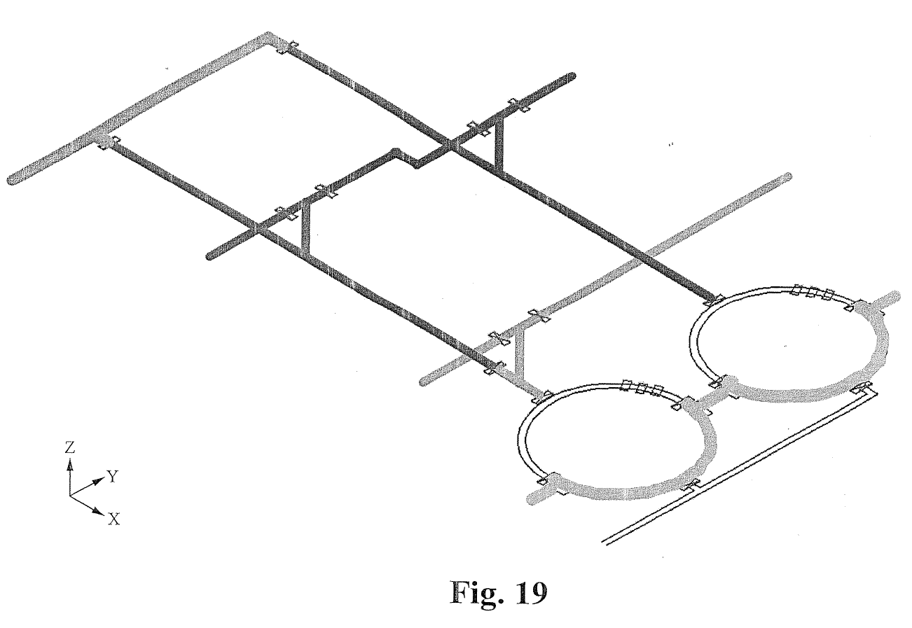

FIG. 19 illustrates the introduction of the dilution buffer for pushing the dilution buffer plus the substrate solution, which is already metered in the second layer parallel processor, into the mixing ring.

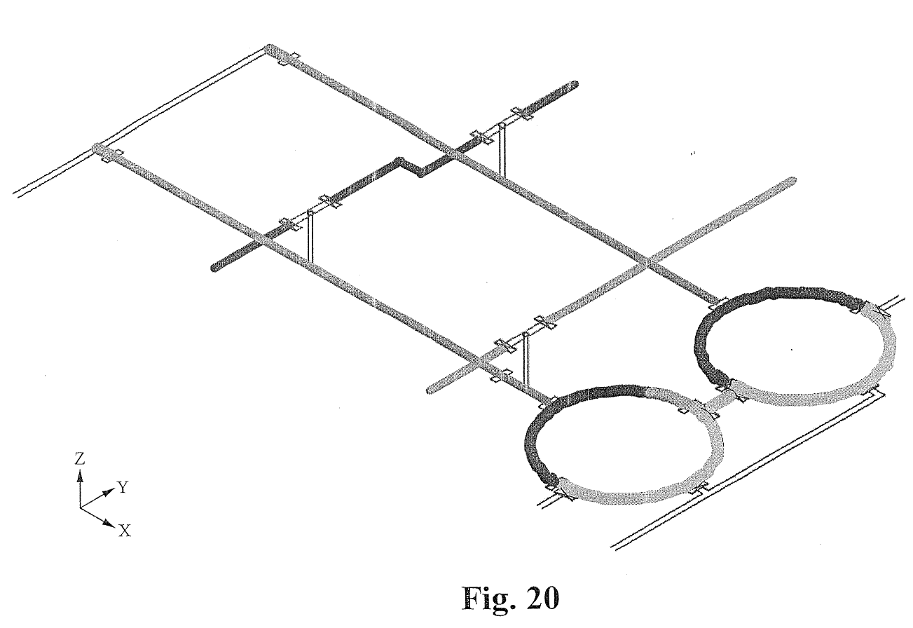

FIG. 20 illustrates the dilution buffer plus the substrate solution, already metered in the second layer parallel processor, being pushed into the mixing ring using the dilution buffer. Note that, as illustrated in FIG. 20, three valves of the processors are open during pushing of the dilution buffer plus substrate solution.

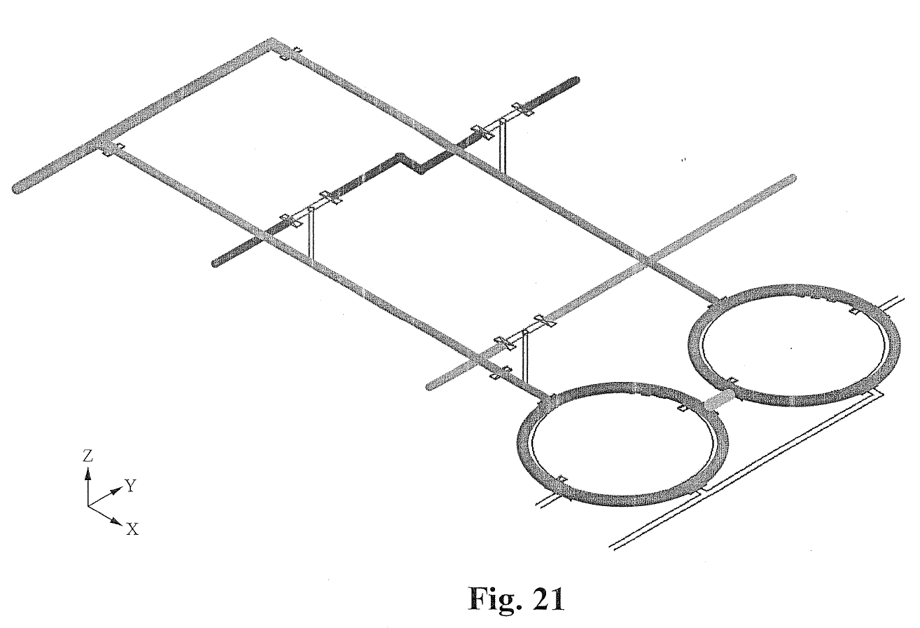

FIG. 21 illustrates that after closing all the valves around the mixing ring, mixing of the dilution buffer plus substrate plus enzyme is accomplished by operating three peristaltic pump valves present into the mixing ring, thus forming the enzyme product.

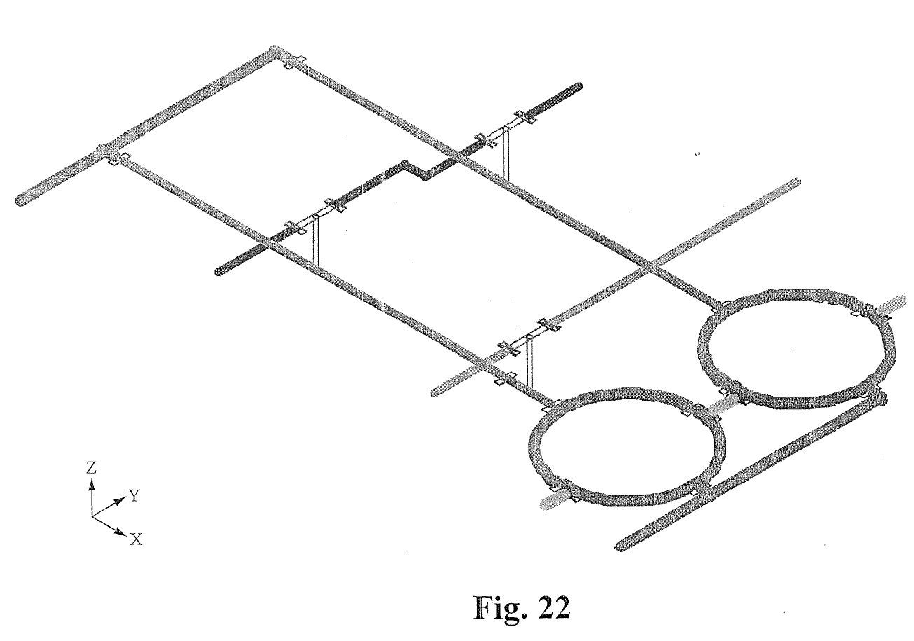

FIG. 22 illustrates that after the optical detection of the enzyme product, the enzyme product is recovered by pushing the enzyme product with the dilution buffer.

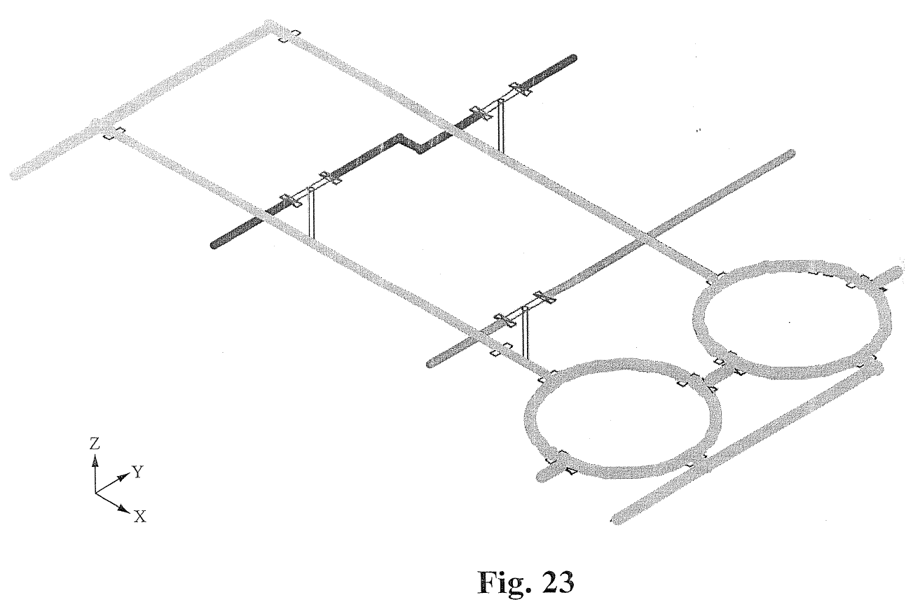

FIG. 23 illustrates that the fluidic processor layer is washed by pushing the wash buffer in all the fluidic processor channels and the mixing ring.

FIG. 24 illustrates an embodiment of the chip design including two layers. This design is realized in two layers from any kind of flexible material such as PDMS. The two layers of the chip design, illustrated in FIG. 24, consist of the top thick slab and the bottom thin film. The top thick slab is used for fluidic sample flow and the bottom thin film is utilized for the control layer. In addition, the chip design consists of complicated and multiple parallel processors with different mixing ratios of reagents such as the dilution buffer (DB) and the substrate (S).

Within the chip design illustrated in FIG. 24, the first layer from the bottom is the Control Layer, “C.” The Control Layer C is used for control of the second layer, known as the fluidic layer “FL.” The second layer FL is intended for supplying, metering and mixing of the reagents in the parallel processors. Hence, the second layer is called the fluidic layer FL.

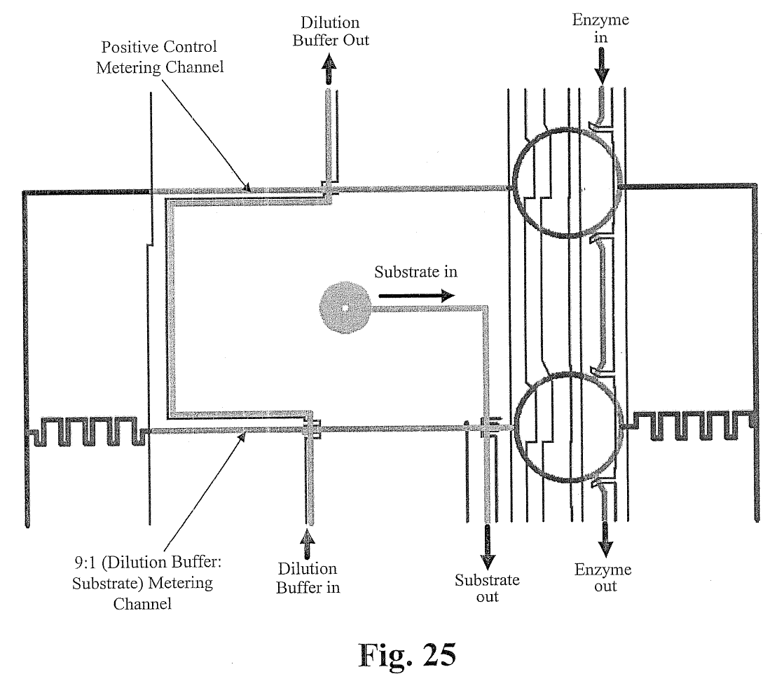

FIG. 25 illustrates a zoomed view of the two layer chip design. To explain the operation of the two layer chip design, two processors having mixing ratios of 10:0 and 9:1 are zoomed out. In the 10:0 processor, there is 100% dilution buffer and 0% substrate. While in the 90:10 processor, there is 90% dilution buffer and 10% substrate. The arrows show direction of movement of reagents into the chip.

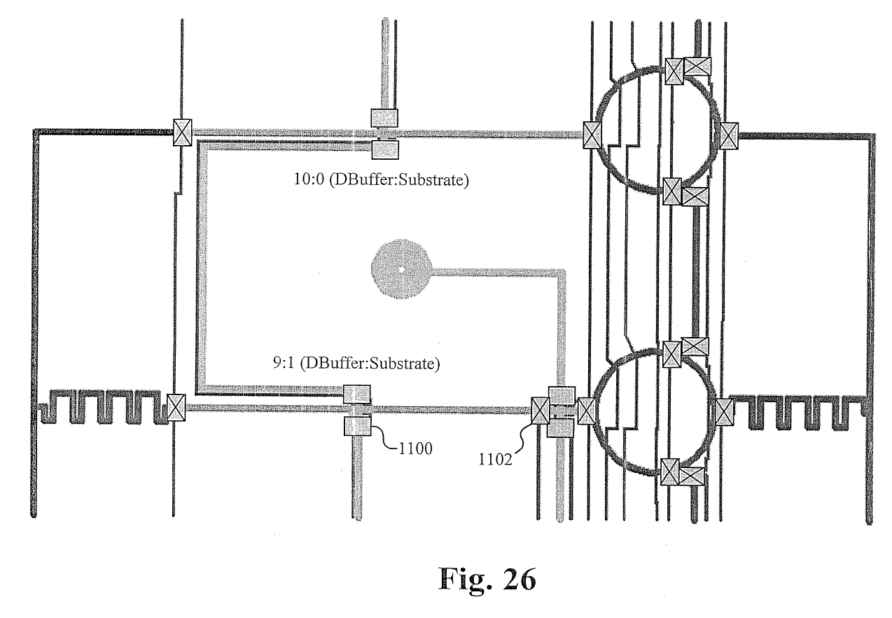

FIG. 26 illustrates a zoomed view of the two layer chip design showing the operation of the two layer chip design. To explain the operation of the two layer chip design, two processors having mixing ratios 10:0 and 9:1, respectively, are zoomed out. In the 10:0 processor, there is 100% dilution buffer and 0% substrate. While in the 90:10 processor, there is 90% dilution buffer and 10% substrate. In FIG. 26, the small hollow rectangles, such as 1100, indicate an open valve, while the crossed rectangles, such as 1102, indicate a closed valve. This designation of open and closed valves is used throughout the Figs. to be discussed below.

As illustrated in FIG. 26, the valves for the dilution buffer and the substrate supply lines are open. Hence, the dilution buffer and the substrate are spread into the dilution buffer and substrate metering channels, respectively.

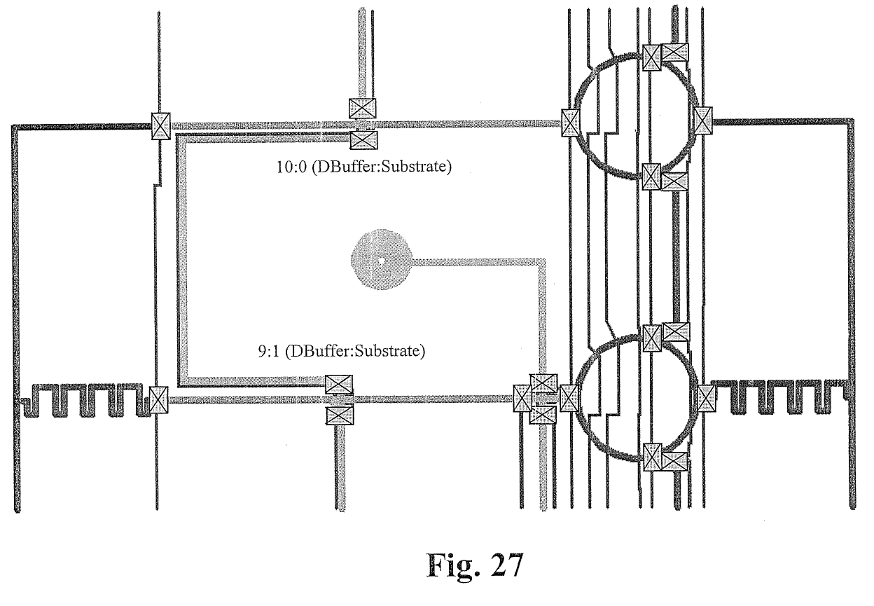

FIG. 27 illustrates the closing of the valves for the dilution buffer and the substrate supply lines. Hence, the correct amount of the dilution buffer and the substrate is metered.

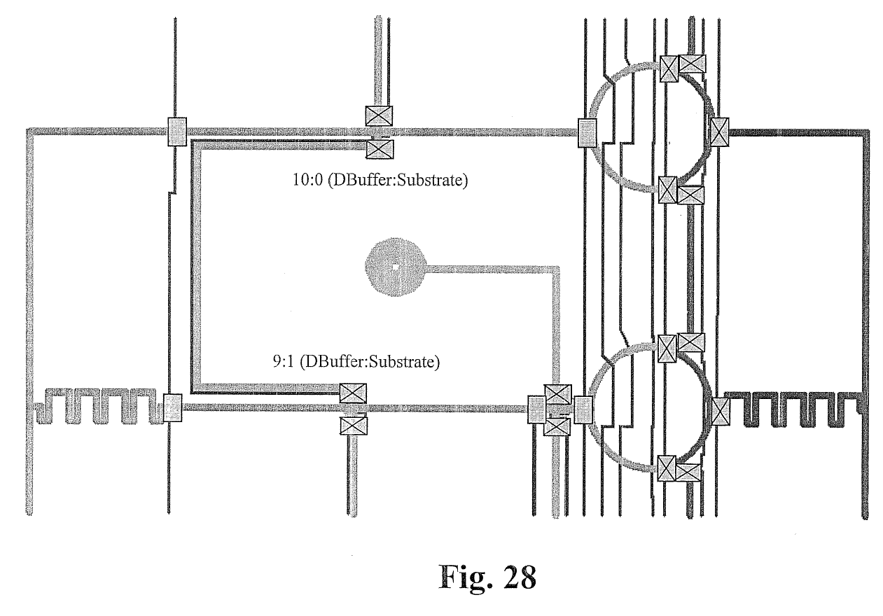

FIG. 28 illustrates the dilution buffer plus substrate solution, already metered in the parallel processor, being pushed into the mixing ring using the dilution buffer. Note that three valves of the processors are open during pushing of the dilution buffer plus substrate solution.

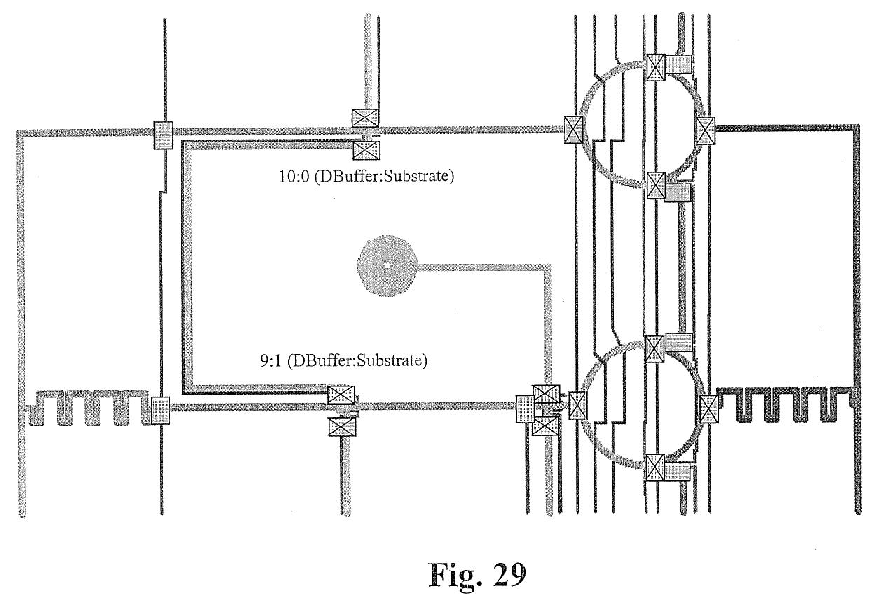

FIG. 29 illustrates that the valves for the enzyme supply lines are open. Hence, the enzyme is spread directly into the enzyme portion in the mixing ring.

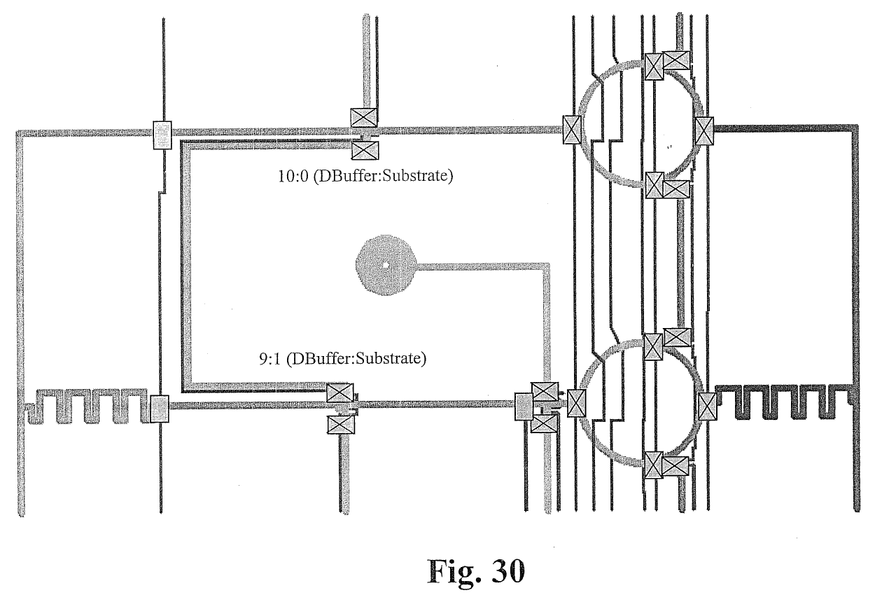

FIG. 30 illustrates that the valves for the enzyme supply lines are closed. Hence, the correct amount of enzyme is metered.

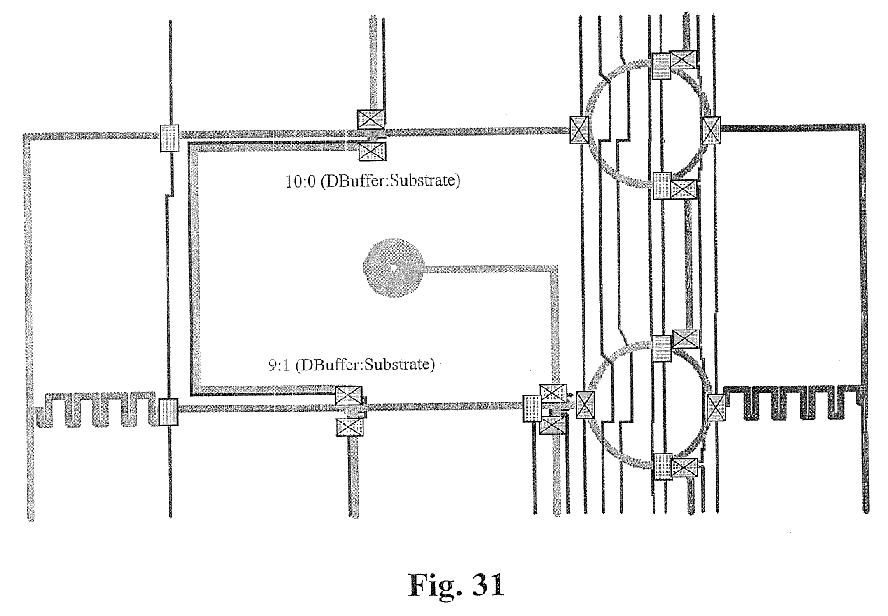

FIG. 31 illustrates the opening of the valve separating the dilution buffer plus substrate and the enzyme in the mixing ring.

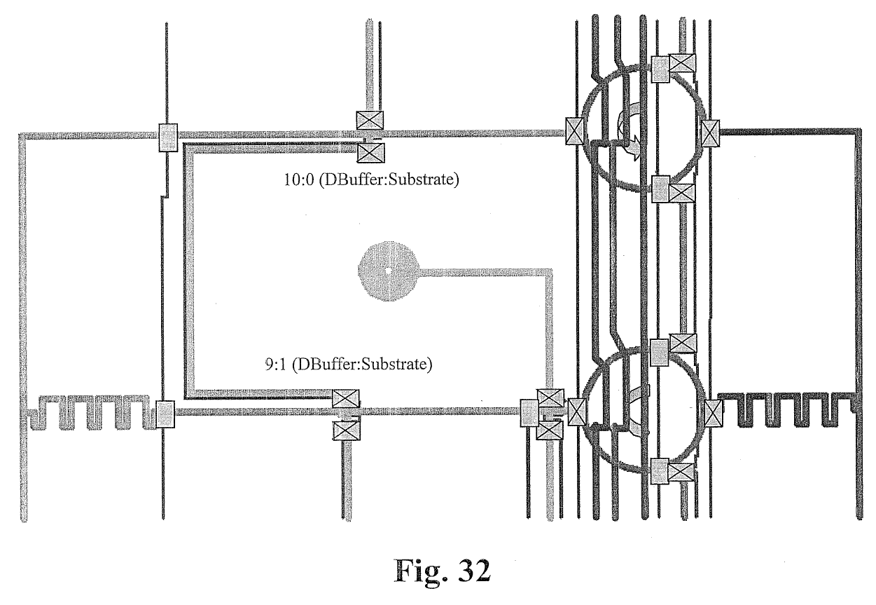

FIG. 32 illustrates that after closing all the valves around the mixing ring, mixing of the dilution buffer plus substrate plus enzyme is accomplished by operating the three peristaltic pump valves present into the mixing ring, thus forming the enzyme product.

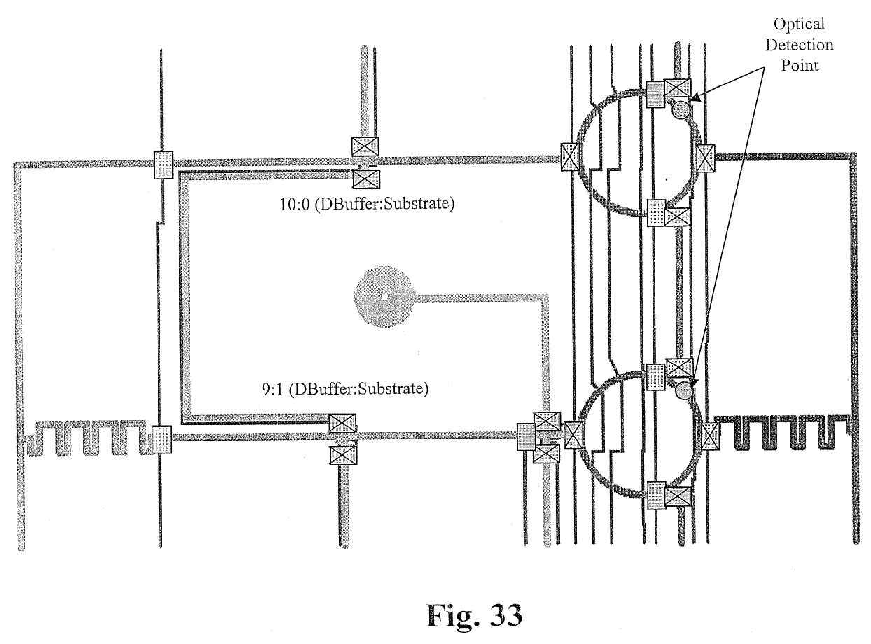

FIG. 33 illustrates optical detection of the enzyme product by using an illuminating top LED layer and collecting the light from the channel into the bottom photo diode array layer.

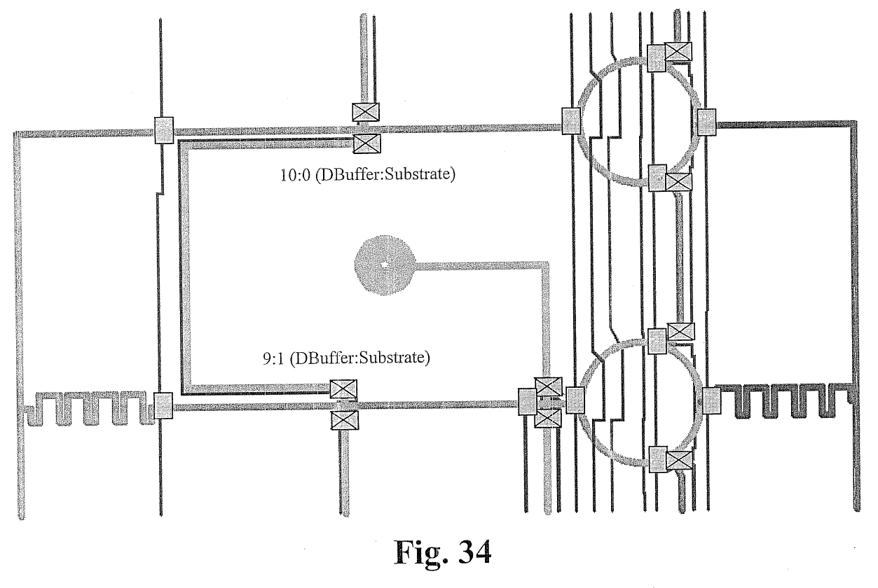

FIG. 34 illustrates that after optical detection of the enzyme product, the enzyme is recovered from all the parallel processors by pushing with the dilution buffer.

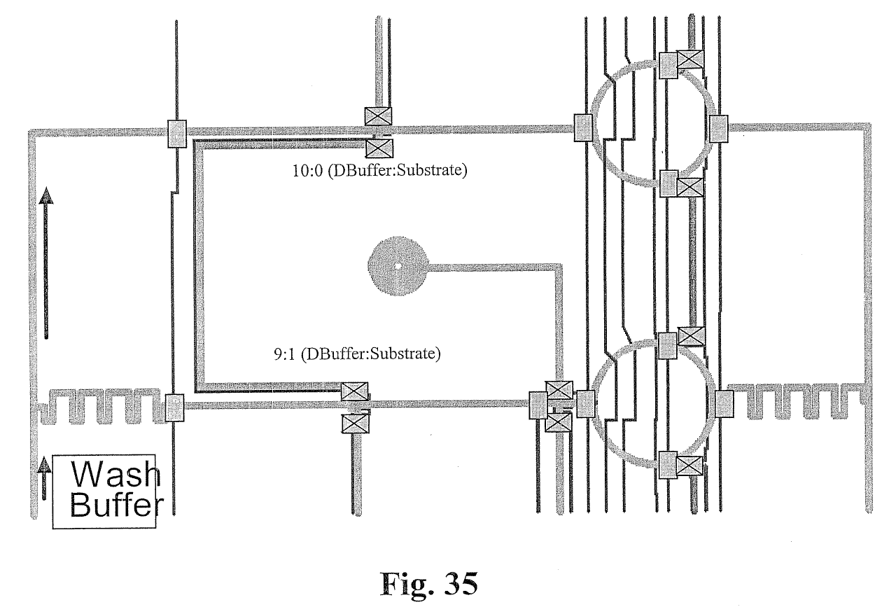

FIG. 35 illustrates washing of the chip by pushing the wash buffer in all the fluidic processor channels and the mixing ring.

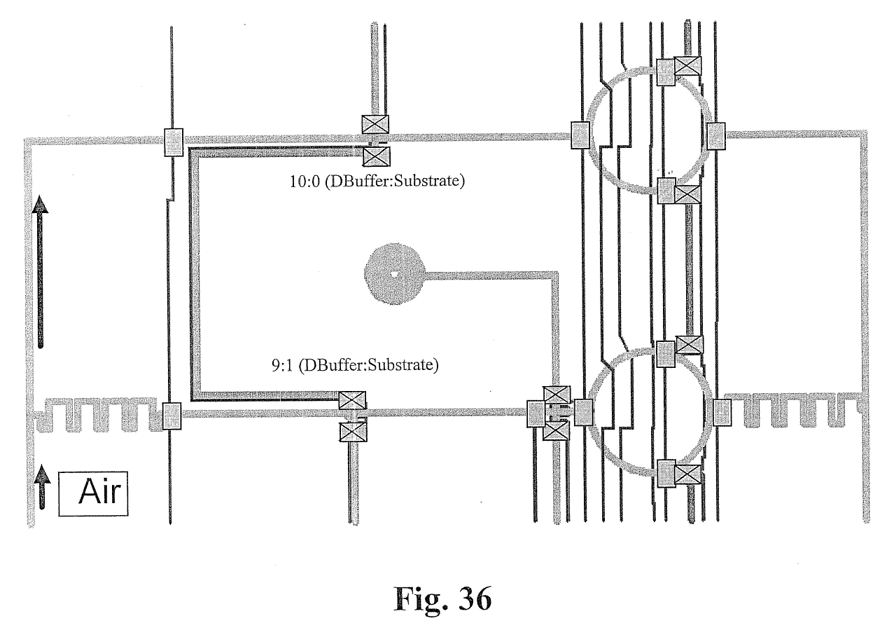

FIG. 36 illustrates drying of the chip by introducing air in all the fluidic processor channels and the mixing ring.

The present invention has been described in terms of specific embodiments incorporating details to facilitate the understanding of the principles of construction and operation of the invention. As such, references herein to specific embodiments and details thereof are not intended to limit the scope of the claims appended hereto. It will be apparent to those skilled in the art that modifications can be made in the embodiments chosen for illustration without departing from the spirit and scope of the invention.

What is claimed is:

1. A system comprising:
a) a controller chip with multiple reaction cells for simultaneously reacting samples of a catalyst in the multiple reaction cells over a range of reaction conditions;
b) an actuator device coupled to the controller chip for mixing the samples of the catalyst with reagents to form a product;
c) a detection unit for detecting kinetic parameters from each of the multiple reaction cells; and
d) a processor coupled to the actuator device for controlling introduction of the catalyst and the reagents into the multiple reaction cells and for collecting and storing the kinetic parameters,
wherein the system characterizes kinetics of the catalyst for the range of conditions.

2. The system of claim 1, further comprising a temperature controller in thermal contact with the controller chip.

3. The system of claim 1, wherein the detection unit comprises one or more of an optical detector, an electrochemical detector and a mass-based cantilever detector.

4. The system of claim 1, wherein the detector is and optical detector that comprises a photodiode array.

5. The system of claim 4, wherein the optical detector further comprises an array of light emitting diodes.

6. The system of claim 1, wherein the controller chip has a parallel reaction cell architecture.

7. The system of claim 1, wherein the controller chip has a circular reaction cell architecture.

8. The system of claim 1, wherein the reaction cells are rotary reaction cells.

9. An optical device comprising a controller chip, the controller chip comprising:
a) multiple optical reactor cells for simultaneously reacting volumes within each of the multiple optical reactor cells; and
b) inlet ports for introducing the volumes into multiple optical reactor cells.

10. The optical device of claim 9, further comprising a temperature controller for moderating temperatures of the optical reactor cells by one or more of thermal contact and optical and radiational heating.

11. The optical device of claim 9, further comprising an optical detector for simultaneously monitoring concentrations of one or more of the reagents as the reagents react within each of the multiple optical reactor cells.

12. The optical device of claim 11, wherein the optical detector comprises a photodiode array.

13. The optical device of claim 11, wherein the optical detector further comprises an array of light emitting diodes.

14. The optical device of claim 9, wherein the multiple optical reactor cells are arranged in a parallel fashion on the controller chip.

15. The optical device of claim 9, wherein the multiple optical reactor cells are arranged in a circular fashion on the controller chip.

16. The optical device of claim 9, wherein the multiple optical reactor cells are rotary reaction cells.

17. The optical device of claim 9, wherein the controller chip is formed from two or more layers.

18. A method of characterizing a kinetic landscape of a catalyst, the method comprising:
a) mixing simultaneously samples of a catalyst in a controller chip under a range of reaction conditions with a substrate to generate a product;
b) measuring kinetic activities of the samples of the catalyst simultaneously; and
c) analyzing the kinetic activities to generate a response curve that characterizes the kinetic landscape of a catalyst.

19. The method of claim 18, wherein the range of reaction conditions includes a range of substrate concentrations and one or more of a range of inhibitor concentrations and a range of pH values.

20. The method of claim 19, wherein measuring the kinetic activities comprises optically detecting a concentration of at least one of the substrate and the product.

21. The method of claim 20, wherein optically detecting comprises measuring an absorption of a light source by at least one of the substrate and product through the controller chip.

22. The method of claim 19, further comprising controlling a temperature value of the controller chip.

23. A controller chip with multiple reaction cells for simultaneously reacting reagents within in the multiple reaction cells over a range of reaction conditions.

24. The controller chip of claim 23, wherein the reaction cells are arranged in parallel on the controller chip.

25. The controller chip of claim 24, wherein the reaction cells are substantially arranged in a circle on the controller chip.

26. A method of characterizing a reaction, the method comprising:
a) mixing simultaneously samples of reagents in controller chip under a range of reaction conditions;
b) measuring activities of the samples simultaneously; and
c) analyzing the activities to generate a response curve that characterizes the reaction

27. The method of claim 26, where the reaction is a reaction selected from the group consisting of a binding reaction, combinatorial reaction and enzymatic reaction.

28. The method of claim 26, wherein the samples of reagents are in one or more of a gaseous state and a liquid state.

29. The method of claim 26, wherein the samples of reagents are biological reagents.

30. The method of claim 29, the biological reagents are selected from the group consisting of bacteria, fungi, viral, richechia and cell biological reagents.
